(19) United States
(12) Patent Application Publication
(10) Pub. No.: US 2026/0069654 A1
(43) Pub. Date: Mar. 12, 2026

(54) NANOAGONIST, AND PREPARATION METHOD AND USE THEREOF

(71) Applicant: CHONGQING UNIVERSITY, Chongqing (CN)

(72) Inventors: Yang LUO, Chongqing (CN); Xingjie LIANG, Chongqing (CN); Guangchao Qing, Chongqing (CN); Yuxuan ZHANG, Chongqing (CN); Yongchao WANG, Chongqing (CN)

(21) Appl. No.: 18/827,759

(22) Filed: Sep. 8, 2024

(51) Int. Cl. A61K 38/16; A61K 47/54; A61P 31/04

(52) U.S. Cl. CPC A61K 38/16; A61K 47/554; A61P 31/04

(57) ABSTRACT

A nanoagonist, and a preparation method and use thereof are provided, belonging to the technical field of nanoscale biomedicine. The nanoagonist is formed by self-assembly of a transformable peptide, where the transformable peptide includes a targeted antimicrobial peptide, a functionalized self-assembling peptide, an FcγR recognition peptide, and a lipase-responsive hydrophobic molecule that are coupled in sequence. The functionalized self-assembling peptide can control the FcγR recognition peptide to flip toward a surface of a target pathogen during secondary self-assembly, and the target pathogen is a pathogen targeted and bound by the targeted antimicrobial peptide. The nanoagonist combines externalization of the FcγR recognition peptide that can be guided during the secondary self-assembly with FcγR-mediated endocytosis, and a nanoagonist is developed for the first time that takes into account both pathogen clearance and host immune function repair.

### REFERENCE TO SEQUENCE LISTING

A computer readable XML file entitled “GWP20240705504”, that was created on Sep. 5, 2024, with a file size of about 3,860 bytes, contains the sequence listing for this application, has been filed with this application, and is hereby incorporated by reference in its entirety.

### TECHNICAL FIELD

The present disclosure belongs to the technical field of nanoscale biomedicine, and in particular relates to a nanoagonist, and a preparation method and use thereof.

### BACKGROUND

Sepsis is a biphasic disease with clinical manifestations divided into an initial inflammatory response and secondary long-term immunosuppression. Anti-inflammatory therapy can alleviate the inflammatory response in an early stage of the sepsis. However, with an increase in the frequency of positive blood cultures and the occurrence of opportunistic microbial infections, immunosuppression and infectious complications in sepsis patients become more prominent. This increases the chances of infection with opportunistic pathogens and reactivated viruses, thus greatly increasing the risk of secondary infection and mortality in patients. Therefore, it is crucial for the treatment of sepsis by repairing the impaired immune function during the immunosuppression phase.

Impaired macrophage function, as one of the causes of impaired immune function and secondary infection in patients with sepsis, can lead to a decrease in the ability of macrophages to secrete pro-inflammatory factors and an increase in the ability to secrete anti-inflammatory factors in a late stage of the sepsis. This mechanism reduces an expression level of human leukocyte antigen-DA on the surface of macrophages and is accompanied by impaired antibacterial ability, causing host immune dysfunction and increasing the risk of secondary infection in patients. Currently, adoptive macrophage therapy can be used to treat sepsis caused by multiple drug-resistant bacterial infections accompanied by immunosuppression. However, a process of exogenously constructing functional macrophages is complex and cannot repair the function of damaged macrophages in patients. Moreover, lysosome-targeted therapy based on metal organic frameworks can ameliorate the damage to an antibacterial ability of macrophages caused by sepsis via releasing calcium and zinc ions to enhance the killing of bacteria hiding in macrophages. However, this therapy cannot effectively respond to secondary infections in a timely manner.

As a result, existing single therapies cannot simultaneously eliminate invading pathogens and repair damaged immune functions, thereby greatly limiting the therapeutic effects of clinical treatment of sepsis and secondary infections.

### SUMMARY

An objective of the present disclosure is to provide a nanoagonist, and a preparation method and use thereof. The present disclosure aims to solve the technical problem that the existing technique cannot simultaneously eliminate invading pathogens and repair damaged immune functions when treating sepsis and secondary infections.

To achieve the above objective, the present disclosure provides the following technical solutions:

A first aspect of the present disclosure provides a nanoagonist. In the present disclosure, a raw material of the nanoagonist includes a transformable peptide; where

the transformable peptide includes a targeted antimicrobial peptide, a functionalized self-assembling peptide, an FcγR recognition peptide, and a lipase-responsive hydrophobic molecule that are coupled in sequence; the functionalized self-assembling peptide controls the FcγR recognition peptide to flip toward a surface of a target pathogen during secondary self-assembly, and the target pathogen is targeted and bound by the targeted antimicrobial peptide.

Preferably, the targeted antimicrobial peptide is used to target and bind to Gram-negative bacteria and/or Gram-positive bacteria.

Preferably, the targeted antimicrobial peptide is selected from the group consisting of UBI₂₉₋₄₁, a targeted antimicrobial peptide I sequence, and targeted antimicrobial peptide II sequence; and

the targeted antimicrobial peptide I sequence is shown in SEQ ID NO: 1, and the targeted antimicrobial peptide II sequence is shown in SEQ ID NO: 2.

Preferably, the FcγR recognition peptide includes tuftsin.

Preferably, the functionalized self-assembling peptide has a sequence shown in SEQ ID NO: 3.

Preferably, the lipase-responsive hydrophobic molecule is one selected from the group consisting of cholesteryl hemisuccinate and monostearyl maleate.

Preferably, the transformable peptide has a chemical structure shown in formula [i]:

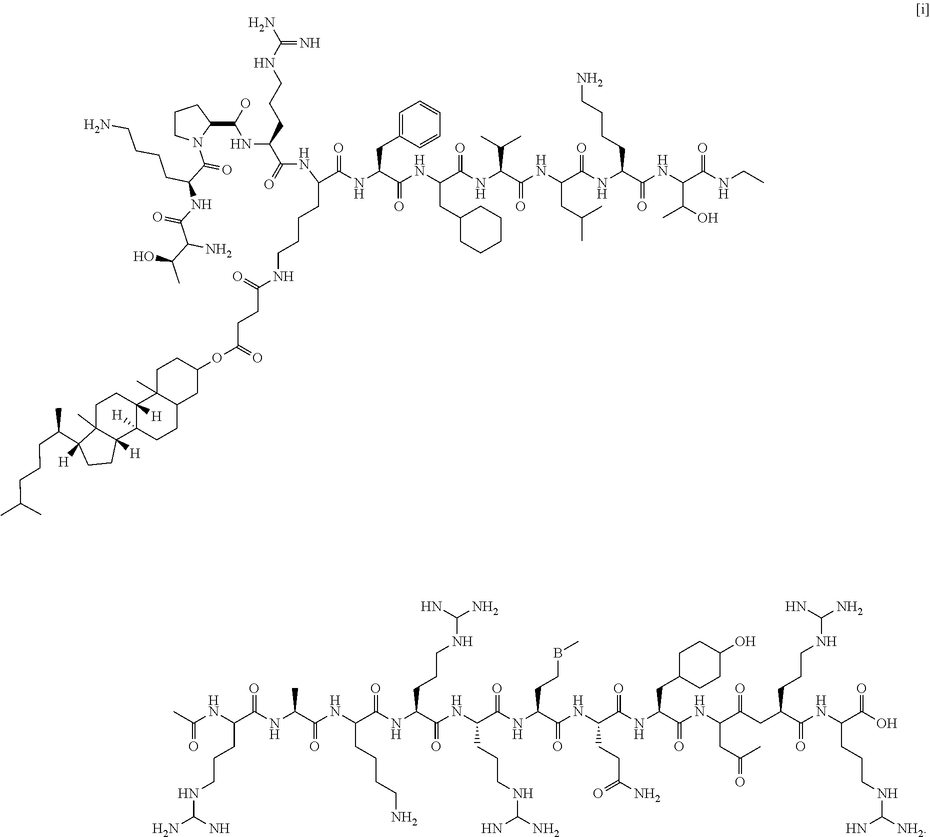

Preferably, the nanoagonist is a spherical nanoparticle with a particle size of 30 nm to 60 nm.

A second aspect of the present disclosure provides a preparation method of the nanoagonist in the first aspect, including the following steps:
synthesizing the transformable peptide;
dissolving the transformable peptide in an organic solvent to obtain a peptide stock solution; and
adding the peptide stock solution into pure water to allow self-assembly to obtain the nanoagonist.

A second aspect of the present disclosure provides use of the nanoagonist in the first aspect in preparation of a drug for treating sepsis and a secondary infection thereof.

Compared with the prior art, the present disclosure at least has the following beneficial effects:

In the present disclosure, the nanoagonist combines externalization of the FcγR recognition peptide that can be guided during the secondary self-assembly with FcγR-mediated endocytosis, and a transformable peptide-based nanoagonist is developed for the first time that takes into account both pathogen control and host immune function repair. The nanoagonist can simultaneously achieve the clearance of invading pathogens and the repair of damaged immune functions in vivo, thus providing a new strategy based on transformable peptide nanotechnology for the effective treatment of sepsis. The nanoagonist is self-assembled by a transformable peptide through sequential coupling of a targeted antimicrobial peptide, a functionalized self-assembling peptide, an FcγR recognition peptide, and a lipase-responsive hydrophobic molecule. On one hand, the nanoagonist has the ability to target and bind to pathogens, such that the nanoagonist can be effectively enriched in the infected tissue and bind to the pathogens, achieving efficient capture, effective inhibition, and response of the infection microenvironment. On the other hand, the secondary assembly behavior and morphology can be controlled based on the hydrophilic and hydrophobic forces in the nanoagonist molecules, such that the FcγR recognition peptide can be turned outward the surface of bacteria and bind to the FcγR on a surface of the macrophage, thereby greatly promoting the macrophage's uptake of the pathogen-peptide complex and promoting the polarization of the macrophage to the M1 type, and finally achieving efficient treatment for sepsis and secondary infections thereof. In addition, the nanoagonist is designed and constructed with humanized peptide molecules as raw materials, has desirable biosafety, and shows a great potential for clinical application.

### BRIEF DESCRIPTION OF THE DRAWINGS

To explain the technical solutions in the examples of the present application clearly, the accompanying drawings required in the examples will be briefly described below. Apparently, the accompanying drawings in the following description show merely some examples of this application, and other drawings may be derived from these accompanying drawings by a person of ordinary skill in the art without creative efforts.

FIG. 1 shows a molecular structure of TP provided in the examples of the present disclosure;

FIG. 2 shows a molecular structure of TP-C1 provided in an example of the present disclosure;

FIG. 3 shows a molecular structure of TP-C2 provided in an example of the present disclosure;

FIG. 4 shows a molecular structure of TP-C3 provided in an example of the present disclosure;

FIG. 5 shows a molecular structure of TP-C4 provided in an example of the present disclosure;

FIG. 6 shows a MALDI-TOF-MS characterization diagram of TP provided in the example of the present disclosure;

FIG. 7 shows a transmission electron microscopy (TEM) image of BactTPNa provided in an example of the present disclosure;

FIG. 8 shows a particle size of BactTPNa provided in an example of the present disclosure;

FIG. 9 shows a MALDI-TOF-MS characterization diagram of BactTPNa+lipase after co-incubation provided in an example of the present disclosure;

FIG. 10 shows a MALDI-TOF-MS characterization diagram of BactTPNa-C2+lipase after co-incubation provided in an example of the present disclosure;

FIG. 11A shows a scanning electron microscopy (SEM) image of S. aureus/P. aeruginosa provided in an example of the present disclosure after co-incubation with PBS, BactTPNa, and BactTPNa-C1; FIG. 11B shows a TEM image of S. aureus/P. aeruginosa provided in an example of the present disclosure after co-incubation with PBS, BactTPNa, and BactTPNa-C1;

FIGS. 12A-12B show experimental results of PBS, BactTPNa, and BactTPNa-C1 in inhibiting the growth of S. aureus/P. aeruginosa provided in an example of the present disclosure, where FIG. 12A is a graph of the number of bacterial colonies obtained after S. aureus/P. aeruginosa is treated with PBS, BactTPNa-C1, and BactTPNa; and FIG. 12B is the statistical data of the number of bacterial colonies corresponding to FIG. 12A;

FIG. 13 shows experimental results of macrophage uptake of bacteria-peptide complexes provided in an example of the present disclosure;

FIGS. 14A-14D show results of BactTPNa promoting macrophage repolarization provided in an example of the present disclosure;

FIGS. 15A-15D show the distribution of BactTPNa and BactTPNa-C1 in the main organs of sepsis mice with lung infection provided in an example of the present disclosure, where FIG. 15A shows the distribution of BactTPNa and BactTPNa-C1 in the main organs; and FIG. 15B shows the average fluorescence intensity statistics of the organs corresponding to FIG. 15A; and

FIGS. 16A-16J show the experimental results of BactTPNa treatment of sepsis mice with lung infection provided in an example of the present disclosure, where FIG. 16A is the survival curve detection result of sepsis mice with lung infection after being treated with different materials;

FIG. 16B is the statistical result of bacterial load in the lungs of sepsis mice with lung infection after being treated with different materials; FIG. 16C is the proportion of F4/80⁺CD80⁺CD86⁺ macrophages in the lungs of sepsis mice with lung infection after being treated with different materials; FIG. 16D is the proportion of F4/80⁺CD206⁺ macrophages in the lungs of sepsis mice with lung infection after being treated with different materials; FIG. 16E is the proportion of CD45⁺CD3⁺CD4⁺ T cells in the lungs of septic mice with lung infection after being treated with different materials; FIG. 16F is the proportion of CD45⁺CD3⁺CD8⁺ T cells in the lungs of sepsis mice with lung infection after being treated with different materials; FIG. 16G is the detection of TNF-α content in the lungs of sepsis mice with lung infection after being treated with different materials; FIG. 16H is the detection of IL-10 content in the lungs of sepsis mice with lung infection after being treated with different materials; FIG. 16I is the proportion of apoptosis of thymic lymphocytes in sepsis mice with lung infection after being treated with different materials; and FIG. 16J is the proportion of CD115⁺CD11b⁺MHC-II⁺ monocytes in the peripheral blood of sepsis mice with lung infection after being treated with different materials.

### DETAILED DESCRIPTION OF THE EMBODIMENTS

The following describes the technical solutions in the embodiments of this application with reference to the accompanying drawings in the embodiments of this application. Apparently, the described embodiments are some rather than all of the embodiments of the present disclosure. All other examples obtained by a person of ordinary skill in the art based on the examples of the present application without creative efforts shall fall within the protection scope of the present application.

In the present application, the term “and/or” describes an association relationship between associated objects, and indicates three types of relationships. For example, “A and/or B” may indicate that A exists alone, A and B coexist, or B exists alone. “A” and “B” each may be singular or plural. The character “/” usually indicates an “or” relationship between associated objects.

The term “at least one” herein refers to one or more, and the term “a plurality of” refers to two or more. The term “at least one of the following items” or similar expression refers to any combination of these items, including any combination of single items or plural items. For example, “at least one of a, b, or c” or “at least one of a, b, and c” can indicate: a, b, c, a-b (namely, a and b), a-c, b-c, or a-b-c, where a, b, and c may each be in a single or plural form.

Those skilled in the art should understand that in the following description of the examples of the present application, the order of sequence numbers of the foregoing processes do not imply the order of execution, and some or all steps may be performed in parallel or sequentially. The order of performing the processes should be determined based on their function and internal logic, and should not constitute any limitation to the implementation process of the embodiments of the present application.

Terms in the embodiments of the present disclosure are merely used to describe the specific embodiments, and are not intended to limit the present disclosure. Unless otherwise specified in the context, words, such as “a”, “the”, and “this”, in a singular form in the embodiments and appended claims of the present disclosure include plural forms.

It should be noted that, in the following description of the embodiments of this application, unless otherwise specified, the technical means involved are all conventional means known in the art. The materials and reagents used are understood according to the general meaning of the art and can be obtained from commercial channels. All the sequences involved are commissioned for synthesis according to the corresponding amino acid sequence information.

In a first aspect, an example of the present disclosure provides a nanoagonist. A raw material of the nanoagonist includes a transformable peptide, which includes a targeted antimicrobial peptide, a functionalized self-assembling peptide, an FcγR recognition peptide, and a lipase-responsive hydrophobic molecule that are coupled in sequence; where the functionalized self-assembling peptide can control the FcγR recognition peptide to flip toward the surface of the target pathogen during secondary self-assembly, and the target pathogen is targeted and bound by the targeted antimicrobial peptide.

It should be understood in the art that the targeted antimicrobial peptide is mainly used to bind to the target head of pathogens, and can be an antimicrobial peptide that can bind to specific proteins on the surface of pathogens, or an antimicrobial peptide that can bind to the surface of pathogens through electrostatic interaction. The targeted antimicrobial peptide of the examples is preferably one of UBI₂₉₋₄₁, a targeted antimicrobial peptide I sequence, and a targeted antimicrobial peptide II sequence; the targeted antimicrobial peptide I sequence is shown in SEQ ID NO: 1; the targeted antimicrobial peptide II sequence is shown in SEQ ID NO: 2. In addition, the sequence of UBI₂₉₋₄₁ is an amino acid sequence known in the art and is not described in detail in the examples.

It should be noted that the pathogens are common bacteria that cause sepsis and its secondary infection, specifically one or more of Gram-negative bacteria and/or Gram-positive bacteria. The Gram-negative bacteria may be Pseudomonas aeruginosa or Escherichia coli; the Gram-positive bacteria may be Staphylococcus aureus or Streptococcus pneumoniae.

It should be understood in the art that the FcγR recognition peptide mainly recognizes FcγR, thereby promoting the binding of the nanoagonist to the FcγR on the surface of macrophages and enhancing the macrophage's ability to take up the pathogen-peptide complexes. The FcγR recognition peptide in the examples is preferably tuftsin, and the sequence of tuftsin is an amino acid sequence known in the art and is not specifically described in the examples.

It should be noted that tuftsin is produced by degradation of the Fc segment of immunoglobulin G in the spleen, and has natural phagocytic function and immunostimulatory activity. The tuftsin can stimulate phagocytosis and chemotaxis by binding to FcγR on the surface of macrophages, which not only significantly enhances the phagocytosis of pathogenic microorganisms by macrophages, but also promotes the release and redistribution of inflammatory cytokines in vivo.

It should be understood in the art that the functionalized self-assembling peptide mainly ligates the targeted antimicrobial peptide and the FcγR recognition peptide to form a peptide segment, and provides power for the secondary self-assembly containing the peptide segment and guides the FcγR recognition peptide to flip outward, thereby causing the FcγR recognition peptide to flip outward to the surface of the target pathogen. The functionalized self-assembling peptide in the examples has a sequence shown in SEQ ID NO: 3.

It should be noted that the sequence shown in SEQ ID NO: 3 is specifically a reverse sequence of the KLVFF peptide derived from β-amyloid protein, and can self-assemble to form a β-folded structure through its own hydrogen bond interaction.

It should be understood in the art that the lipase-responsive hydrophobic molecule mainly provides a hydrophobic core for the assembly of the transformable peptide and provides bacterial lipase responsiveness to the nanoagonist. The lipase-responsive hydrophobic molecule in the examples is preferably one of cholesteryl hemisuccinate and monostearyl maleate. Both the cholesteryl hemisuccinate and monostearyl maleate contain a lipid bond, which can be broken under the action of bacterial lipase, thereby achieving bacterial lipase response of the nanoagonist.

In the examples of the present disclosure, when the pathogenesis of severe sepsis and/or secondary infection is deeply understood, it is found that the clinical treatment of secondary infection generally faces the difficulty in simultaneously eliminating pathogens and repairing the host's impaired immune function through a single therapy. To solve this problem, a transformable nanoagonist is prepared by combining nanotechnology with bio-immunity technology. The nanoagonist is formed by self-assembly of a transformable peptide which is sequentially coupled with a targeted antimicrobial peptide, a functionalized self-assembling peptide, an FcγR recognition peptide, and a lipase-responsive hydrophobic molecule, such that the nanoagonist has the following characteristics and advantages:
1) In the examples of the present disclosure, the nanoagonist has the ability to target and bind to pathogens, which can effectively enrich the nanoagonist in infected tissues and bind to and encapsulate pathogens, thereby achieving efficient capture, effective inhibition, and response of the infection microenvironment.
2) In the examples of the present disclosure, the nanoagonist has both hydrophilic and hydrophobic forces and non-covalent forces, such that the assembly behavior and morphology of the nanoagonist can be effectively controlled based on the hydrophilic and non-covalent forces (specifically, the nanoagonist can remove the hydrophobic core under the action of high concentration of bacterial lipase at the infection site, and a tuftsin unit inside the nanoagonist can be turned outward toward the bacterial surface with the help of the secondary assembly of the functionalized self-assembling peptide). This enables the exposed tuftsin effectively binds to the FcγR on the surface of the macrophage, thus greatly improving the macrophage's uptake of the pathogen-peptide complexes and promoting the polarization of the macrophage to the M1 type.
3) Based on the interaction between the FcγR recognition peptide and the FcγR on the macrophages, the nanoagonist can promote macrophage phagocytosis and polarization toward M1 type, thereby achieving the restoration of host immune function. As a result, the nanoagonist has the dual effects of clearing pathogens and restoring host immune function, and can be used to prepare drugs for treating sepsis and its secondary infections.
4) In the present disclosure, the nanoagonist is designed and constructed using humanized peptide molecules as raw materials, has desirable biosafety, and shows great clinical application potential and excellent clinical translational application effect.

The present disclosure is based on the preferred composition and specific amino acid sequence of the transformable peptide described above. Accordingly, the transformable peptide has a chemical structure shown in formula [i]:

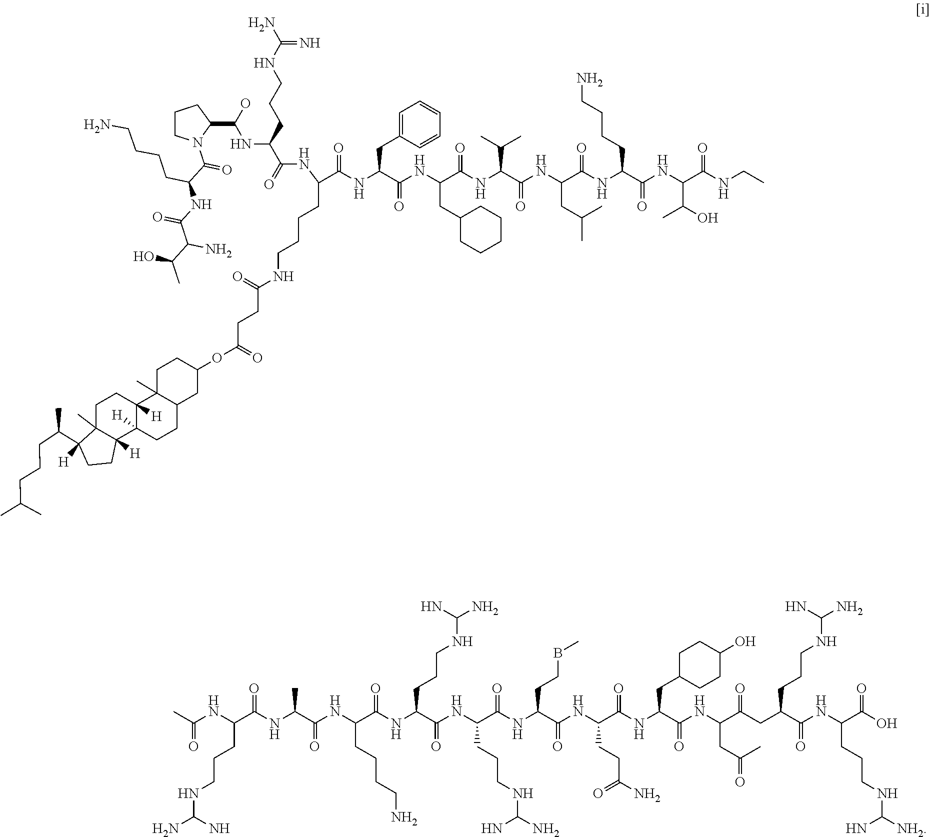

In some specific examples, the nanoagonist is a spherical nanoparticle with a particle size of 30 nm to 60 nm and is monodisperse. In general, the targeted antimicrobial peptide is located outside the spherical nanoparticle, and the FcγR recognition peptide is located inside the spherical nanoparticle. The specific particle size may be 30 nm, 35 nm, 40 nm, 45 nm, 50 nm, 55 nm, or 60 nm, without particular limitation.

In a second aspect, an example of the present disclosure provides a preparation method of the nanoagonist, including steps S10 to S30:
S10, synthesizing the transformable peptide;
S20, dissolving the transformable peptide in an organic solvent to obtain a peptide stock solution; and
S30, adding the peptide stock solution into pure water to allow self-assembly to obtain the nanoagonist.

In the examples of the present disclosure, the transformable peptide is preferably synthesized by a solid phase synthesis method known in the art, and a specific synthesis method includes S101 to S111:
S101, immersing a Wang resin loaded with a first amino acid carrying a protecting group in DMF to allow soaking and swelling;
S102, immersing a resulting swollen Wang resin in a deprotecting agent to allow deprotection, and washing the Wang resin alternately with DCM and DMF after a protecting group Fmoc is removed to obtain a Wang resin coupling I; where the deprotection is successful as confirmed by a ninhydrin method, and the deprotecting agent is a mixture of DMF, piperazine, 1,8-diazabicyclo[5.4.0]undec-7-ene (DBU), and formic acid at a mass ratio of 92:5:2:1;
S103, dissolving a condensation agent (benzotriazole-N,N,N′,N′-tetramethyluronium hexafluorophosphate. HBTU) and amino acid in a coupling agent, and adding a resulting mixture into the Wang resin coupling I to allow reaction to obtain a Wang resin coupling II, where a ninhydrin method test proves that the next amino acid is successfully coupled;
S104, coupling to the last amino acid according to the operations of S102 to S103 to obtain a Wang resin coupling III;
S105, immersing the Wang resin coupling III in a hydrazine hydrate solution to allow deprotection, and adding a coupling agent solution containing HBTU and a lipase-responsive hydrophobic molecule after a protecting group Dde is removed, and conducting a reaction for 12 h to 24 h to obtain a Wang resin coupling IV;
S106, immersing the Wang resin coupling IV in a deprotecting agent to allow deprotection, and washing the Wang resin coupling IV alternately with DCM and DMF after the protecting group Fmoc is removed to obtain a Wang resin coupling V;
S107, eluting the Wang resin coupling V with methanol and draining for later use; and
S108, immersing the Wang resin coupling V in a lysis solution to allow lysis, collecting a resulting solution and removing the solvent, and subjecting an obtained product to washing with glacial diethyl ether and vacuum drying to obtain the transformable peptide, which is stored in a refrigerator at 4° C.; where the lysis solution is a mixture of trifluoroacetic acid (TFA), water, and triisopropylsilane at a volume ratio of 95:2.5:2.5.

The technical solutions of the present disclosure will be further described below in conjunction with specific examples.

### Example 1

This example provided a preparation method of a nanoagonist (BactTPNa), including steps S10 to S30:
S10, a transformable peptide (TP) was synthesized by solid phase synthesis.

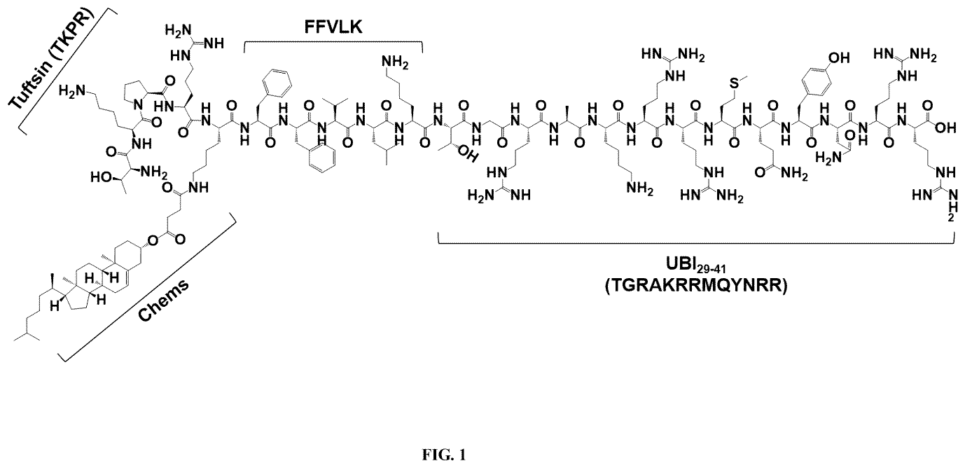

As shown in FIG. 1, the transformable peptide (TP) was a molecular structure formed by sequentially coupling UBI₂₉₋₄₁, FFVLK, tuftsin, and cholesteryl hemisuccinate (Chems), and a synthesis method included steps S101 to S108:
S101, 300 mg to 500 mg of a Wang resin loaded with arginine was immersed in 8 mL to 10 mL of DMF and allowed to swell for 3 h to 6 h;
S102, a resulting swollen Wang resin was immersed in 8 mL to 10 mL of a deprotecting agent to allow deprotection for 15 min to 30 min, and the Wang resin alternately was washed with DCM and DMF after a protecting group Fmoc was removed to obtain a Wang resin coupling I; where the deprotection was successful as confirmed by a ninhydrin method, and the deprotecting agent was a mixture of DMF, piperazine, 1,8-diazabicyclo[5.4.0]undec-7-ene (DBU), and formic acid at a mass ratio of 92:5:2:1;
S103, a condensing agent HBTU equivalent to 10 times a molar amount of arginine and the next amino acid were dissolved in 8 mL to 10 mL of a coupling agent in sequence, shaken for 15 min to 30 min, a resulting mixture was added into the Wang resin coupling I to allow reaction for 45 min to 90 min to obtain a Wang resin coupling II, where the ninhydrin method test proved that the amino acid coupling was successful, and the coupling agent was a mixture of DMF and N-methylmorpholine at a volume ratio of 95:5;
S104, coupling to the last amino acid according to the operations of S102 to S103 to obtain a Wang resin coupling III;
S105, the Wang resin coupling III was immersed in 8 mL to 10 mL of a 2% hydrazine hydrate solution to allow deprotection 3 min×3 times, and a coupling agent solution containing HBTU and cholesteryl hemisuccinate (Chems) were added after a protecting group Dde was removed, and a reaction was conducted for 12 h to 24 h to obtain a Wang resin coupling IV;
S106, the Wang resin coupling IV was dissolved in a deprotecting agent to allow deprotection for 15 min to 30 min, and the Wang resin coupling IV was washed alternately with DCM and DMF after the protecting group Fmoc was removed to obtain a Wang resin coupling V;
S107, the Wang resin coupling V was eluted with 6 mL to 8 mL of methanol three times and drained for later use; and
S108, the Wang resin coupling V was immersed in 2 mL to 3 mL of a lysis solution to allow lysis in an ice water bath at 300 rpm to 400 rpm for 2 h to 3 h, a resulting solution was collected and the solvent was removed by a rotary evaporator, and an obtained product was subjected to washing with glacial diethyl ether three times and vacuum drying to obtain the transformable peptide, which is stored in a refrigerator at 4° C. for later use; where the lysis solution was a mixture of TFA, water, and triisopropylsilane at a volume ratio of 95:2.5:2.5.
S20, a peptide stock solution was prepared.

The transformable peptide was dissolved in DMSO to obtain a peptide stock solution.
S30, self-assembly formation was conducted.

The peptide stock solution was quickly added into pure water and ultrasonically treated for 30 min, and then allowed to stand at room temperature for 2 h to 4 h to allow self-assembly to obtain the nanoagonist (BactTPNa).

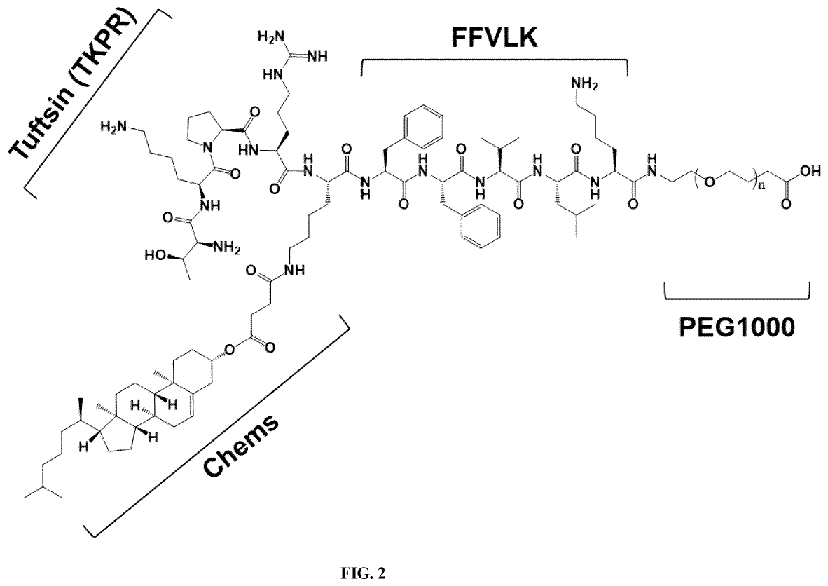

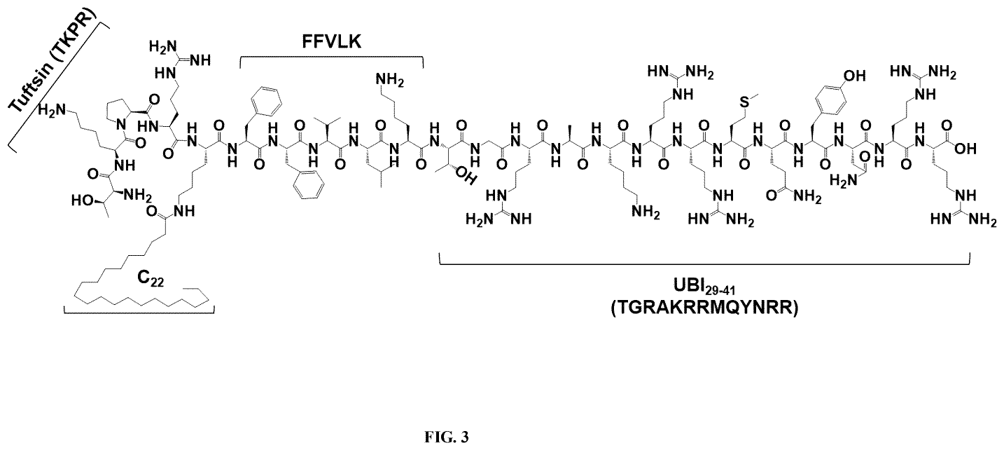

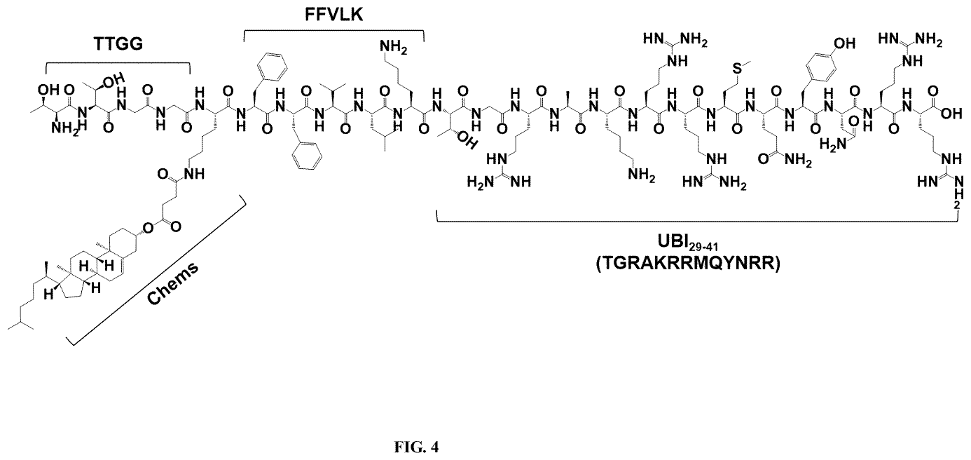

Meanwhile, in this example, peptides (TP-C1), (TP-C2), (TP-C3), and (TP-C4), as well as nanoagonists (BactTPNa-C1), (BactTPNa-C2), (BactTPNa-C3), and (BactTPNa-C4) were prepared as comparative examples, where the comparative examples had the following differences from the examples, and the remaining structures and synthesis methods were the same as those in the examples, specifically:
as shown in FIG. 2, the TP-C1 was a peptide structure formed by replacing UBI₂₉₋₄₁ in TP with PEG1000, followed by sequential coupling of PEG1000, FFVLK, tuftsin, and Chems;
as shown in FIG. 3, the TP-C2 was a peptide structure formed by replacing Chems in TP with docosanoic acid (C22), followed by sequential coupling of UBI₂₉₋₄₁, FFVLK, tuftsin, and C22;
as shown in FIG. 4, the TP-C3 was a peptide structure formed by replacing tuftsin in TP with TTGG, followed by sequential coupling of UBI₂₉₋₄₁, FFVLK, TTGG, and Chems; and
as shown in FIG. 5, the TP-C4 was a peptide structure formed by replacing FFVLK in TP with GGAAK, followed by sequential coupling of UBI₂₉₋₄₁, GGAAK, TTGG, and Chems.

In order to verify the structural characteristics and actual performance of TP and BactTPNa prepared in Example 1, structural characterization and experimental analysis were conducted on TP and BactTPNa, specifically:

### 1. MALDI-TOF-MS Characterization of TP

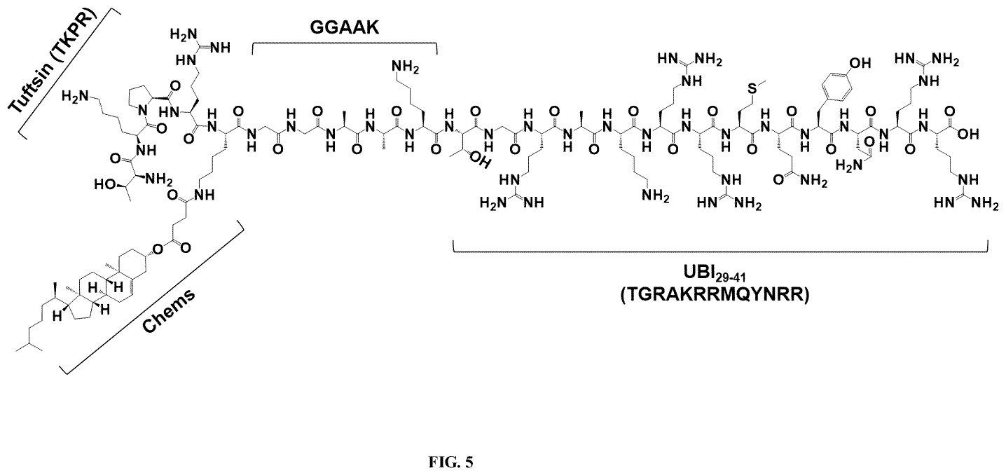

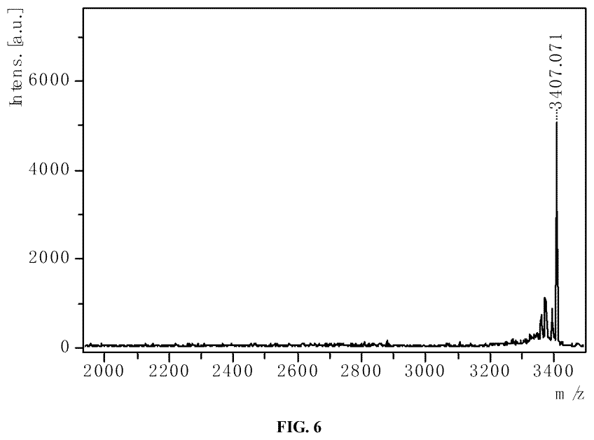

In the present disclosure, TP was characterized by MALDI-TOF-MS, and the results were shown in FIG. 6. FIG. 6 showed a MALDI-TOF-MS characterization diagram of TP.

As shown in FIG. 6, the molecular weight of TP was measured to be 3407.071 g/mol, which was consistent with the theoretical molecular ion peak of the corresponding peptide+the molecular ion peak of two hydrogen ions, indicating that TP was successfully synthesized.

### 2. TEM Characterization of BactTPNa

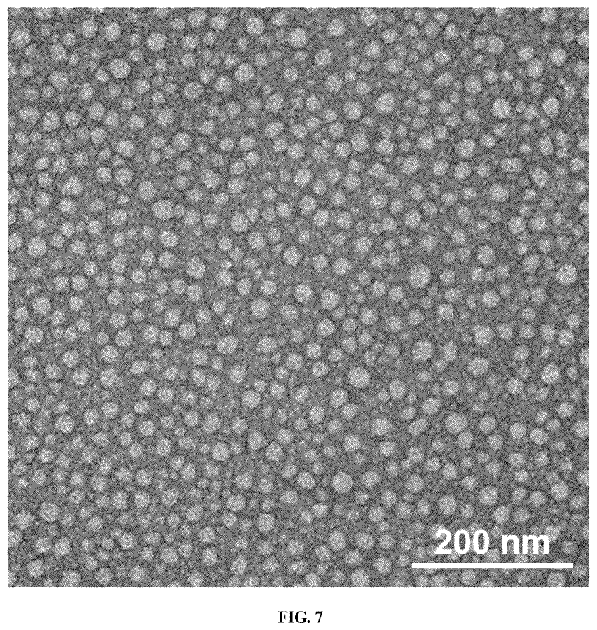

In the present disclosure, BactTPNa was characterized by TEM, and the results were shown in FIG. 7. FIG. 7 showed a TEM image of BactTPNa.

According to FIG. 7, the particle size of BactTPNa prepared in Example 1 was uniform and monodisperse.

### 3. Dynamic Light Scattering Characterization of BactTPNa

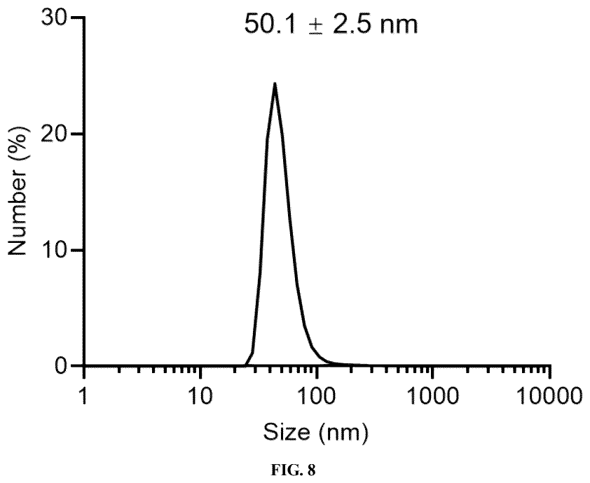

In the present disclosure, BactTPNa was characterized by dynamic light scattering, and the results were shown in FIG. 8. FIG. 8 showed a particle size of BactTPNa.

According to FIG. 8, the particle size of BactTPNa prepared in Example 1 was not greater than 50 nm.

### 4. Bacterial Lipase Responsiveness Evaluation Experiment of BactTPNa

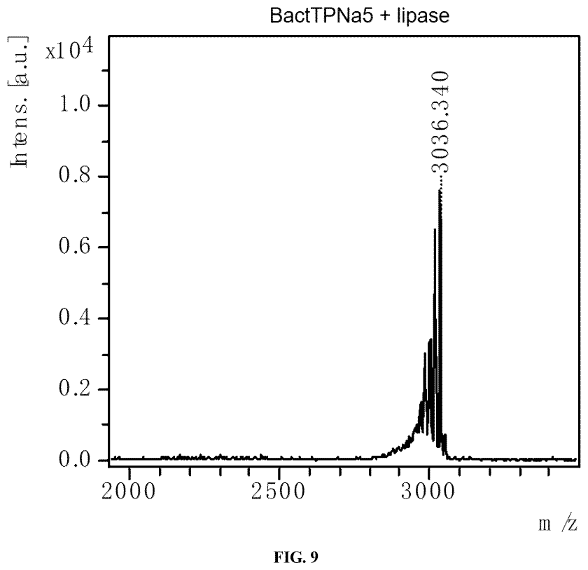

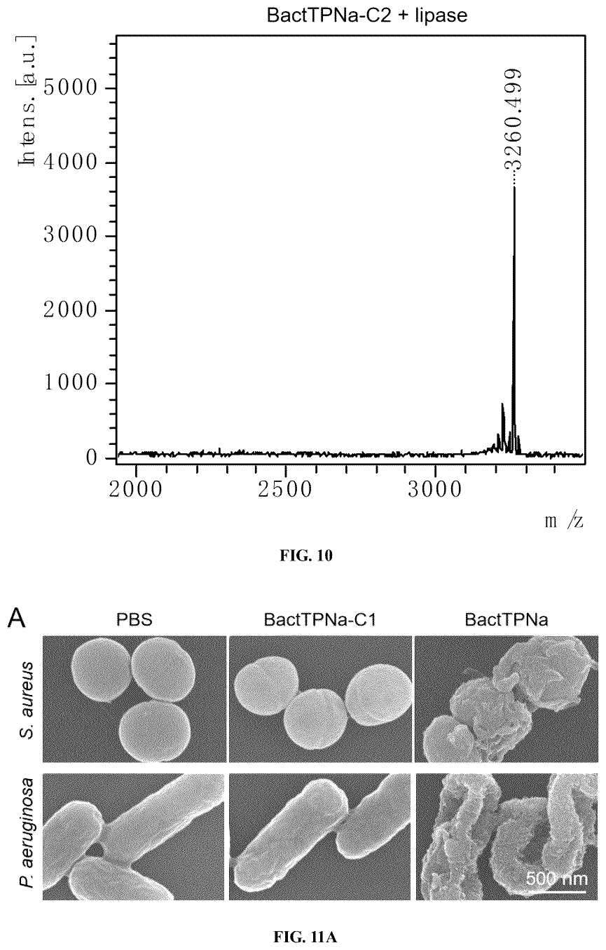

The evaluation method of this experiment included: 50 μL of 10 mg/mL bacterial lipase solution was added to BactTPNa and BactTPNa-C2 solutions with a concentration of 30 μM, shaken at 37° C. for 12 h, and a molecular weight of the resulting solution was detected by time-of-flight mass spectrometry. The results were shown in FIG. 9 and FIG. 10. FIG. 9 showed a MALDI-TOF-MS characterization diagram of BactTPNa+lipase after co-incubation; FIG. 10 showed a MALDI-TOF-MS characterization diagram of BactTPNa-C2+lipase after co-incubation.

As shown in FIG. 9 and FIG. 10, the molecular weight of BactTPNa treated with bacterial lipase was consistent with the molecular weight after Chems removal, indicating that BactTPNa prepared in Example 1 could respond to bacterial lipase to remove Chems; while the BactTPNa-C2 did not have this function.

### 5. Experimental Evaluation of BactTPNa's Ability to Bind Bacteria

The evaluation method of this experiment included: after culturing S. aureus and P. aeruginosa to the logarithmic growth phase, the two bacteria with OD₆₀₀=0.1 were added into BactTPNa and BactTPNa-C1 solutions, respectively, shaken at 37° C. for 30 min, centrifuged at 3,000 g for 5 min, and washed with PBS three times. The obtained product was fixated with 2.5% glutaraldehyde solution overnight and dehydrated with gradient concentrations of ethanol aqueous solution, and then resuspended in ethanol. 5 μL of the resuspension was dropped onto the surface of the silicon wafer. After drying, it was sprayed with gold and observed using a scanning electron microscope. The results were shown in FIG. 11A. FIG. 11A showed a SEM image of S. aureus/P. aeruginosa after co-incubation with PBS, BactTPNa, and BactTPNa-C1.

After culturing S. aureus and P. aeruginosa to the logarithmic growth phase, the two bacteria with OD₆₀₀=0.1 were added into BactTPNa and BactTPNa-C1 solutions, respectively, shaken at 37° C. for 30 min, centrifuged at 3,000 g for 5 min, and washed with PBS three times. The obtained product was fixated with 1% osmium acid fixative at room temperature for 2 h, and then dehydrated using gradient concentrations of ethanol aqueous solution, a mixed solution of 100% ethanol and 100% acetone (v:v=1:1), and 100% acetone. The samples were then immersed in acetone-epoxy resin embedding agent (Epon 812) solutions with a volume ratio of 1:1 and 1:2 for 1 h separately. The resulting product was infiltrated overnight with acetone-epoxy embedding agent solution and 100% epoxy embedding agent in a volume ratio of 1:3. The impregnated samples were placed in constant-temperature incubators at 37° C., 45° C., and 70° C., respectively, and each polymerized for 24 h to form an embedded block. The embedded block was cut into ultrathin sections using an ultrathin slicer and placed on an electron microscope grid. After staining with 3% uranyl acetate-lead citrate, the sections were observed and recorded using TEM. The results were shown in FIG. 11B. FIG. 11B showed a TEM image of S. aureus/P. aeruginosa after co-incubation with PBS, BactTPNa, and BactTPNa-C1.

As shown in FIG. 11, BactTPNa could effectively bind to the surface of S. aureus and P. aeruginosa, indicating that BactTPNa had desirable bacterial binding ability; after co-incubation with BactTPNa-C1, the surface morphology of S. aureus and P. aeruginosa was not significantly different from that of the bacteria in the PBS group, indicating that BactTPNa-C1 did not have bacterial binding ability.

### 6. Evaluation Experiment of BactTPNa Inhibiting Bacterial Growth

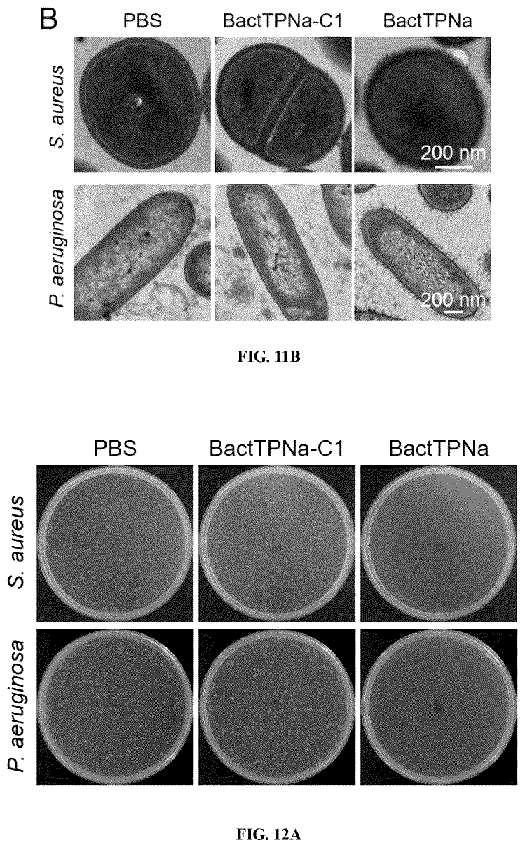

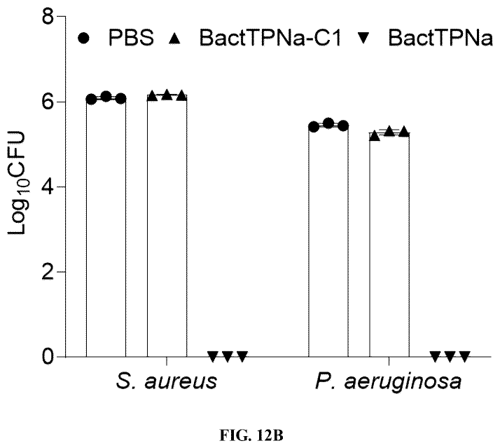

The evaluation method of this experiment included: after S. aureus and P. aeruginosa were cultured to the logarithmic growth phase, the two bacteria with OD₆₀₀=0.1 were taken, and incubated with 1 mL of 30 μM BactTPNa and BactTPNa-C1 for 1 h, centrifuged at 8,000 g for 5 min, the supernatant was discarded, and a product was washed twice with PBS. The product was bacteria bound to BactTPNa or BactTPNa-C1, thereby obtaining a bacteria-peptide complex. The bacteria-peptide complex was dispersed in 1 mL of beef extract peptone medium, and a resulting solution was further diluted 10,000 times. 50 μL of a diluted solution was placed in solid beef extract peptone medium and plated and cultured overnight. Finally, the number of bacterial colonies in each plate was recorded, and the results were shown in FIGS. 12A-12B. FIGS. 12A-12B showed experimental results of PBS, BactTPNa, and BactTPNa-C1 in inhibiting the growth of S. aureus/P. aeruginosa.

FIG. 12A was a graph of the number of bacterial colonies obtained after S. aureus/P. aeruginosa was treated with PBS, BactTPNa-C1, and BactTPNa; and FIG. 12B was the statistical data of the number of bacterial colonies corresponding to FIG. 12A.

As shown in FIG. 12A and FIG. 12B, BactTPNa could effectively inhibit the growth of S. aureus and P. aeruginosa, while BactTPNa-C1 did not have the ability to inhibit the growth of S. aureus and P. aeruginosa.

7. Bacterial Evaluation after Macrophage Uptake and Co-Incubation with BactTPNa

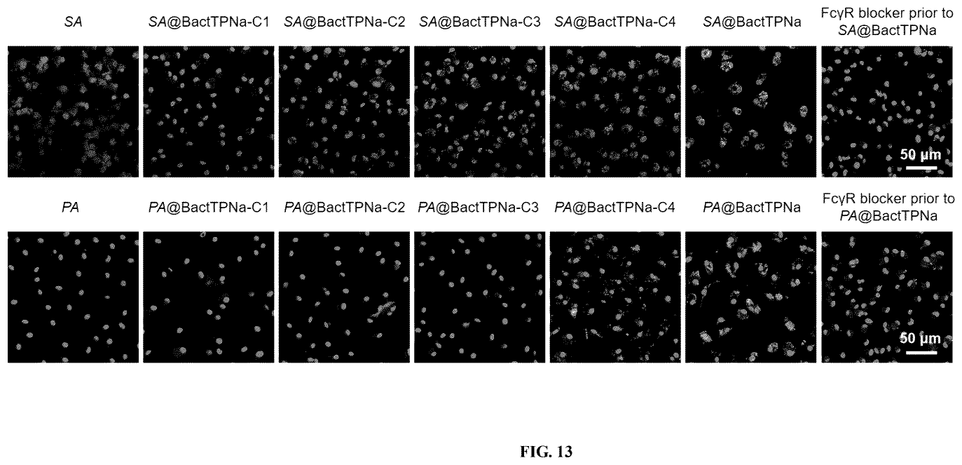

The evaluation method of this experiment included: FITC-labeled S. aureus and P. aeruginosa were incubated with Cy5.5-labeled BactTPNa, BactTPNa-C1, BactTPNa-C2, BactTPNa-C3, and BactTPNa-C4 for 12 h, and the resulting bacteria-peptide complexes SA@BactTPNas and PA@BactTPNas were dispersed in 300 μL of PBS. 50 μL of the SA@BactTPNas or PA@BactTPNas was added into an eight-well dish seeded with M2 BMDM and incubated in a cell culture incubator at 37° C. and 5% CO₂ for 20 min. The medium was discarded and the bacterial cells were carefully washed twice with PBS. Then, 200 μL of medium containing 3 drops of Hochest/mL was added to each well in sequence, and incubated in the cell culture incubator for another 20 min. The supernatant was discarded, the bacterial cells were washed twice with PBS, 200 μL of medium was added, and the samples in the eight-well dish were observed and recorded using a single-photon laser confocal imaging system. The results were shown in FIG. 13. FIG. 13 showed experimental results of macrophage uptake of bacteria-peptide complexes.

As shown in FIG. 13, in the treatment groups of SA@BactTPNa5 and PA@BactTPNa5, the green fluorescence labeling the two bacteria and the red fluorescence labeling the nanoagonist showed desirable co-localization, and both were in the cytoplasm. Blocking FcγR might significantly reduce the uptake of SA@BactTPNa5 and PA@BactTPNa5 by BMDM, indicating that BMDM could take up more SA@BactTPNa5 and PA@BactTPNa5 and its uptake amount was significantly higher than that of other groups, and the uptake process was FcγR-dependent.

### 8. Experimental Evaluation of BactTPNa Promoting Macrophage Repolarization

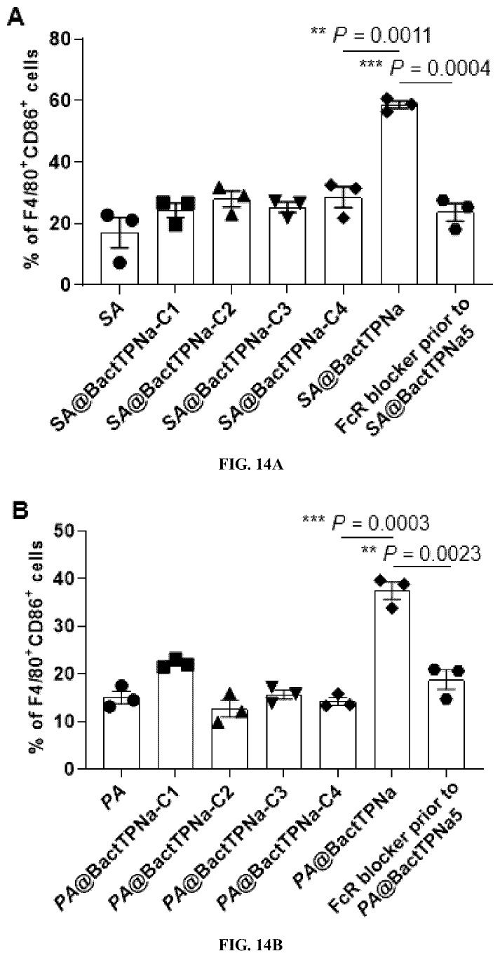

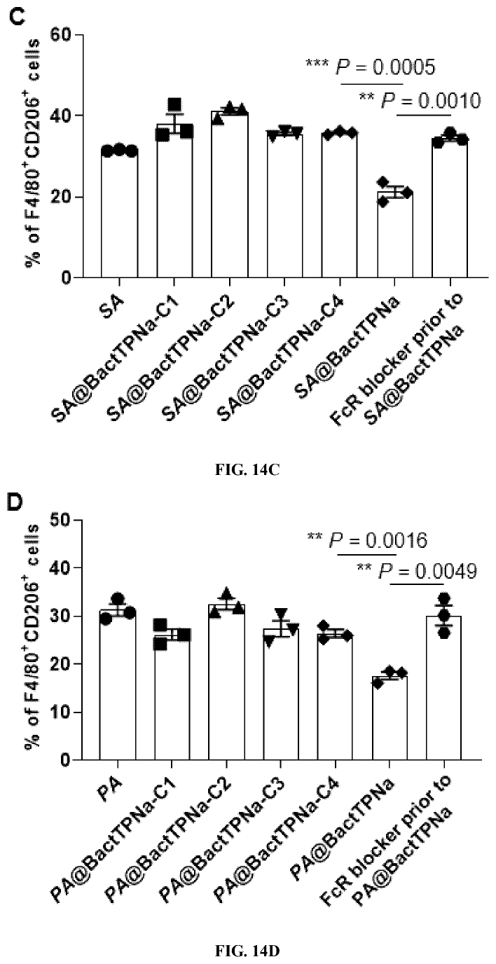

The evaluation method of this experiment included: after obtaining the unlabeled bacteria-peptide complexes SA@BactTPNas and PA@BactTPNas according to the method of Experiment 7, 100 μL was added to a 12-well plate inoculated with M2-type BMDM and incubated in a cell culture incubator at 37° C. and 5% CO₂ for 4 h. The bacterial cells in the wells were washed twice with PBS, added with 500 μL of trypsin/well, and then incubated in a cell culture incubator for 5 min, and 500 μL of complete medium was added to terminate the trypsin digestion. The digested cells were collected and centrifuged at 500 g for 5 min. The supernatant was discarded, and 100 μL of cell staining buffer containing FITC anti-mouse CD206 (1:100), PE anti-mouse F4/80 (1:100), APC anti-mouse CD86 (1:100), and BV421 anti-mouse CD80 (1:100) was added, and the bacterial cells were stained in a 4° C. refrigerator in the dark for 30 min. After centrifugation at 500 g for 5 min, the supernatant was discarded, the bacterial cells were washed once with PBS, and then dispersed in 400 μL of PBS. The expression of CD80, CD86, and CD206 on the surface of the obtained cells was detected by flow cytometry, and the results were shown in FIGS. 14A-14D.

FIGS. 14A-14D showed results of BactTPNa promoting macrophage repolarization.

FIG. 14A showed the percentage of F4/80⁺CD86⁺ macrophages after co-incubation with SA@BactTPNas; FIG. 14B showed the percentage of F4/80⁺CD86⁺ macrophages after co-incubation with PA@BactTPNas; FIG. 14C showed the percentage of F4/80⁺CD206⁺ macrophages after co-incubation with SA@BactTPNas; FIG. 14D showed the percentage of F4/80⁺CD206⁺ macrophages after co-incubation with PA@BactTPNas.

According to FIG. 14A to FIG. 14D, after co-incubation with SA@BactTPNa-C1-4, the proportions of BMDM (F4/80⁺CD86⁺ macrophages) with high expression of co-stimulatory factor CD86 was 24.3±4.1%, 28.0±4.5%, 25.3±3.0% and 28.6±5.9%, respectively; after co-incubation with SA@BactTPNa and PA@BactTPNa, the proportion of F4/80⁺CD86⁺ BMDM increased significantly, reaching 58.6±1.0% and 37.4±3.1%, respectively, which was close to or more than twice that of the other four groups. In addition, after treatment with SA@BactTPNa and PA@BactTPNa, the proportion of BMDM (F4/80⁺CD206⁺ macrophages) with high expression of CD206 decreased to 21.1±2.4% and 17.6±1.4%, respectively, indicating that the SA@BactTPNa and PA@BactTPNa could effectively promote the polarization of M2 BMDM to M1.

### 9. Characterization of the Distribution of BactTPNa In Vivo

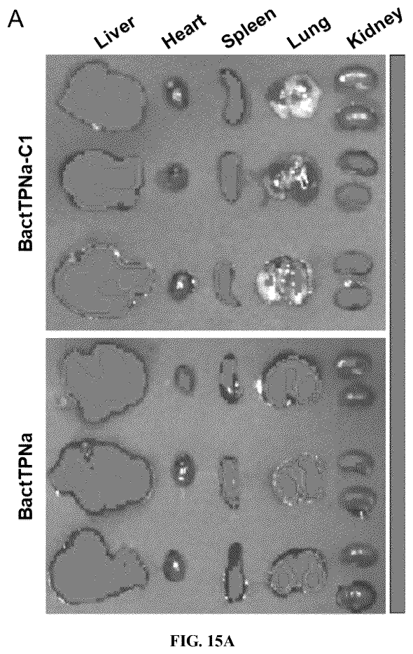

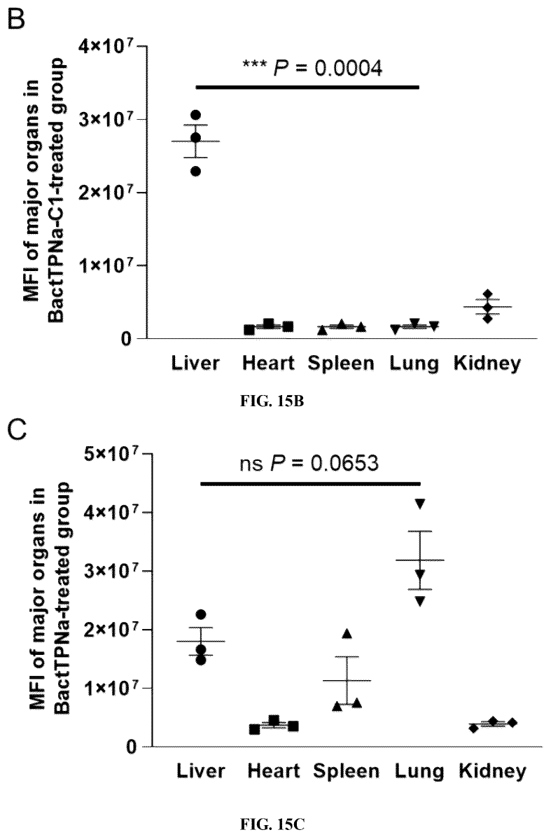

The specific characterization methods included: a mouse model of sepsis induced by multi-bacterial infection accompanied by immunosuppression was constructed through cecal slurry (CS) injection, and based on this, a lung infection model was constructed using P. aeruginosa. 6 healthy C57BL/6n male mice were randomly divided into two groups, with 3 mice in each group. Each group of mice was intraperitoneally injected with 100 μL of CS at an injection dose of 0.4 g/kg. 72 h after injection, the mice were anesthetized with isoflurane, and 30 μL of bacterial solution with OD₆₀₀=0.6 was instilled into each mouse through the trachea. After 12 h, 100 μL of 200 μM Cy5-labeled BactTPNa and BactTPNa-C1 were injected through the tail vein; after 24 h, the mice were euthanized, and their hearts, livers, spleens, lungs, and kidneys were removed. The fluorescence intensity of each organ was recorded using a small animal optical 3D in vivo imaging system, and the results were shown in FIGS. 15A-15D. FIGS. 15A-15D showed the distribution of BactTPNa and BactTPNa-C1 in the main organs of sepsis mice with lung infection. Specifically:

FIG. 15A showed the distribution of BactTPNa and BactTPNa-C1 in the main organs; and FIG. 15B showed the average fluorescence intensity statistics of the organs corresponding to FIG. 15A.

As shown in FIGS. 15A-15D, the BactTPNa was mainly accumulated in the liver and lungs, a small amount in the spleen, and extremely little in the heart and kidneys. The BactTPNa-C1 was mainly enriched in the liver, and less enriched in other organs. At the same time, the average fluorescence intensity in the lungs of mice injected with BactTPNa was significantly higher than that of mice injected with BactTPNa-C1, indicating that BactTPNa had a stronger ability to target bacterial infection sites. In addition, although a large amount of BactTPNa was also enriched in the liver, unlike BactTPNa-C1, BactTPNa could be more effectively enriched in the infected lungs of mice. This proved that the presence of UBI₂₉₋₄₁ gave BactTPNa better bacterial targeting ability, which might be the main reason why BactTPNa could be enriched in large quantities in infected lungs.

10. Evaluation of BactTPNa in Treating Sepsis Mice with Lung Infection

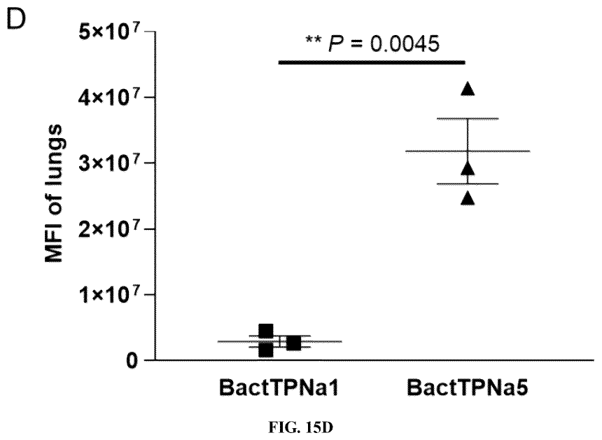

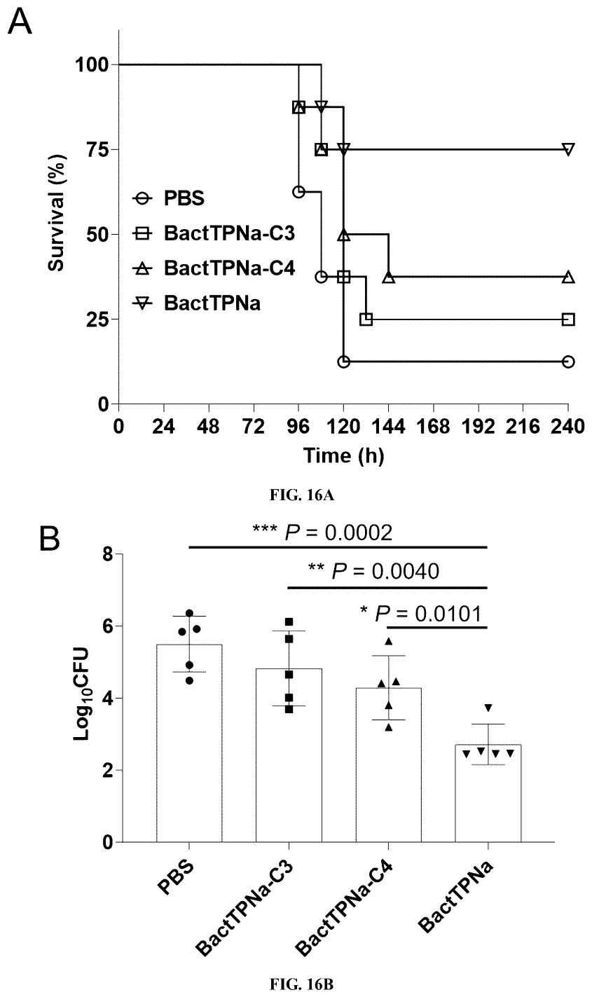

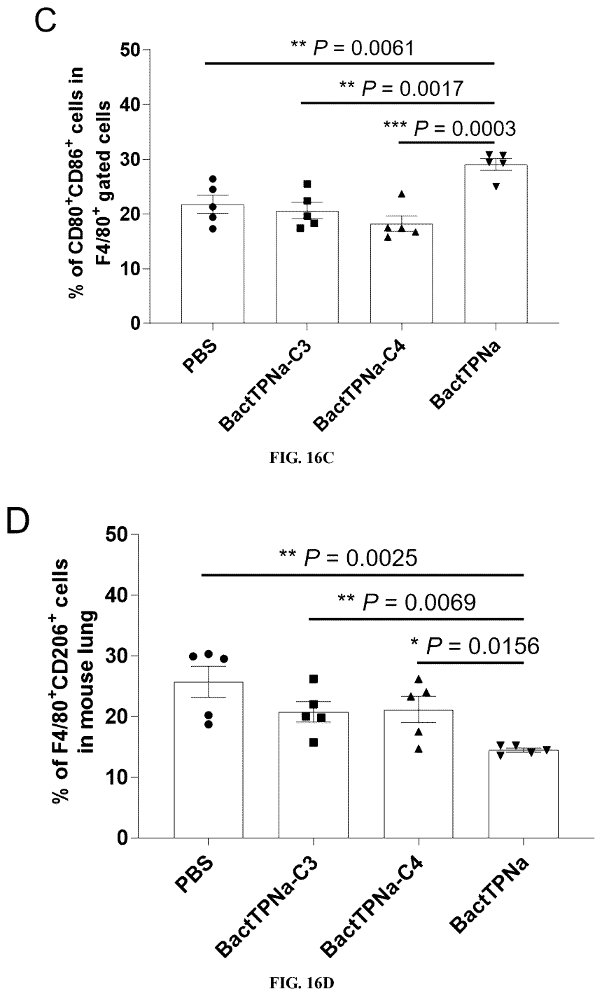

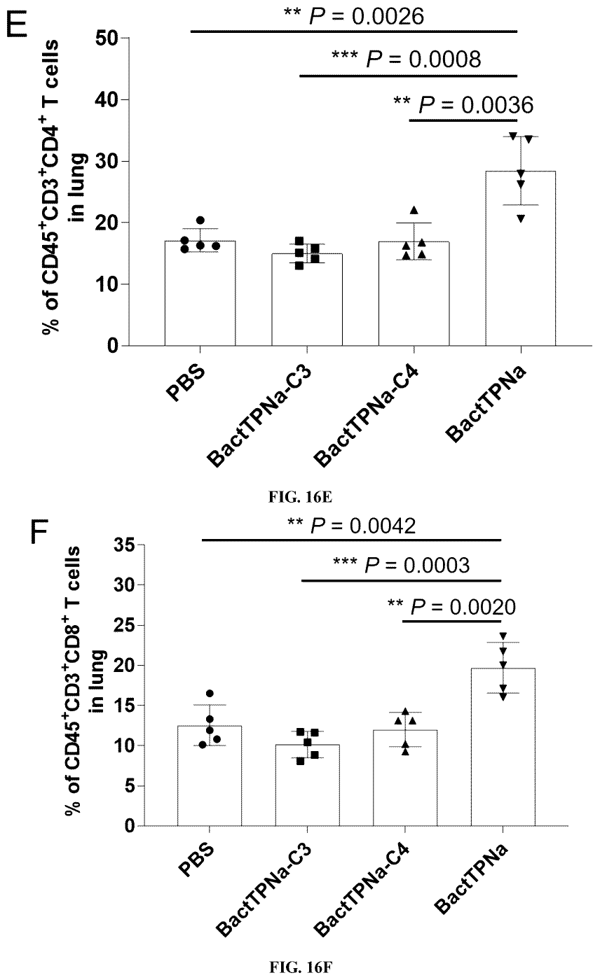

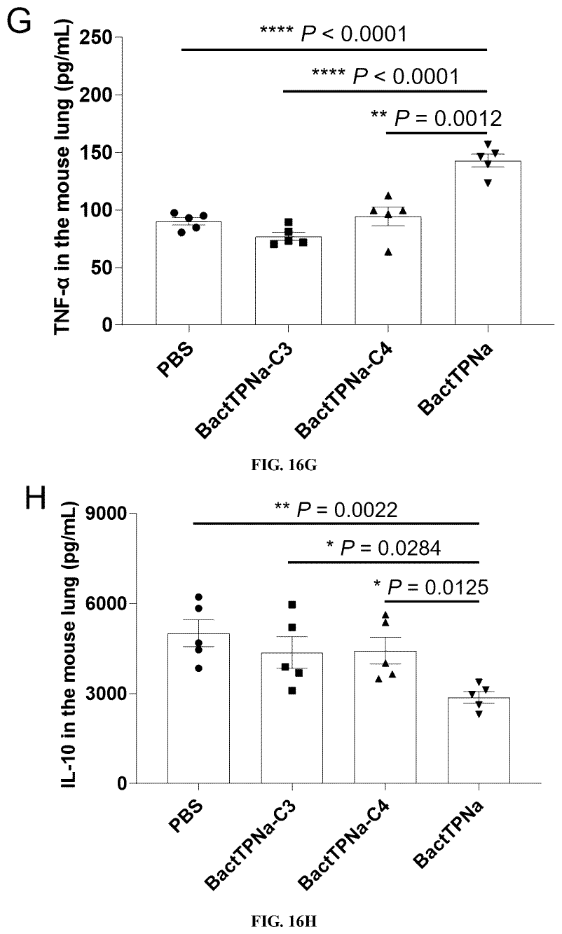

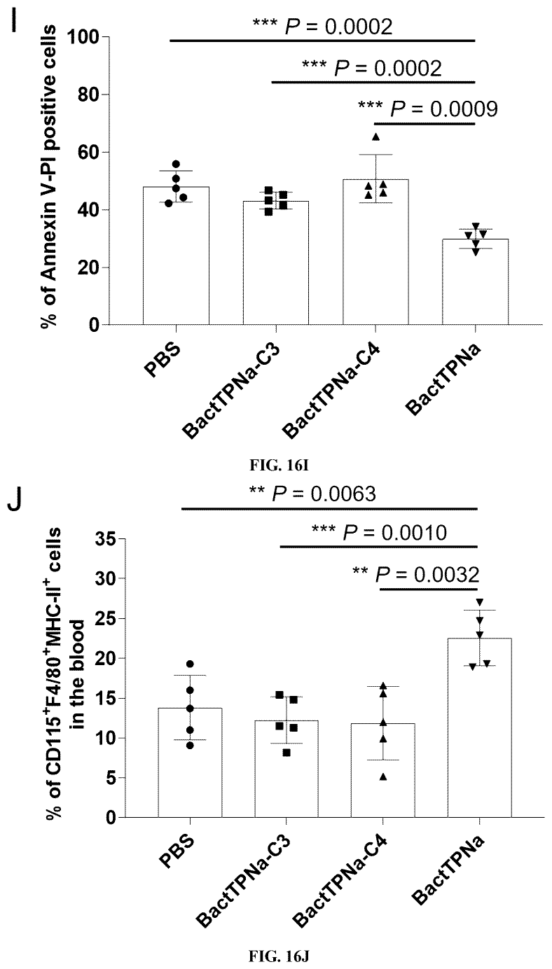

The evaluation method of this experiment included: a mouse model of sepsis induced by multi-bacterial infection accompanied by immunosuppression was constructed through CS injection, and based on this, a lung infection model with P. aeruginosa was constructed to obtain a mouse model of sepsis accompanied by lung infection. The sepsis mice with lung infection were randomly divided into 5 groups, with 8 mice in each group. 12 h after perfusion of P. aeruginosa, each group of mice was injected with 100 μL of 200 μM BactTPNa, BactTPNa-C3, and BactTPNa-C4 via the tail vein. While mice injected with 100 μL PBS served as a control. The body weight and number of surviving mice were recorded until the 10th day after CS injection. Similarly, on the third day after the injection of the nanoagonist, the bacterial load in the lung tissue was evaluated by plate coating method; the macrophage phenotype and lymphocyte status in the lung tissue were detected by flow cytometry. Similarly, the expression of MHC-II on the surface of peripheral blood monocytes and the apoptosis of thymic lymphocytes were evaluated by flow cytometry, and the contents of TNF-α and IL-10 in the lung tissue were determined by ELISA. The results were shown in FIGS. 16A-16J. FIGS. 16A-16J showed the experimental results of BactTPNa treatment of sepsis mice with lung infection. Specifically:

FIG. 16A was the survival curve detection result of sepsis mice with lung infection after being treated with different materials; FIG. 16B was the statistical result of bacterial load in the lungs of sepsis mice with lung infection after being treated with different materials; FIG. 16C was the proportion of F4/80⁺CD80⁺CD86⁺ macrophages in the lungs of sepsis mice with lung infection after being treated with different materials; FIG. 16D was the proportion of F4/80⁺CD206⁺ macrophages in the lungs of sepsis mice with lung infection after being treated with different materials; FIG. 16E was the proportion of CD45⁺CD3⁺CD4⁺ T cells in the lungs of septic mice with lung infection after being treated with different materials; FIG. 16F was the proportion of CD45⁺CD3⁺CD8⁺ T cells in the lungs of sepsis mice with lung infection after being treated with different materials; FIG. 16G was the detection of TNF-α content in the lungs of sepsis mice with lung infection after being treated with different materials; FIG. 16H was the detection of IL-10 content in the lungs of sepsis mice with lung infection after being treated with different materials; FIG. 16I was the proportion of apoptosis of thymic lymphocytes in sepsis mice with lung infection after being treated with different materials; and FIG. 16J was the proportion of CD115⁺CD11b⁺MHC-II⁺ monocytes in the peripheral blood of sepsis mice with lung infection after being treated with different materials.

As shown in FIGS. 16A-16J, BactTPNa treatment could effectively inhibit lung infection in mice and improve the survival rate of mice. Flow cytometry results showed that BactTPNa treatment significantly increased the proportion of F4/80⁺CD80⁺CD86⁺ macrophages, CD4 T, and CD8 T cells in the lungs and reduced the proportion of F4/80⁺CD206⁺ macrophages. In addition, BactTPNa treatment could promote the expression of MHC-II on the surface of monocytes in the peripheral blood of mice and inhibit the apoptosis of thymus and spleen cells. ELISA results showed that after BactTPNa treatment, the expression of TNF-α in the lungs of mice increased and was significantly higher than that of other treatment groups, while the expression of IL-10 was significantly reduced. All these indicated that BactTPNa could effectively inhibit lung infection in mice and repair damaged immune function, thereby improving the survival rate of sepsis mice.

The embodiments in this specification are described in a progressive manner. For same or similar parts between embodiments, reference may be made to each other. Each embodiment focuses on a difference from other embodiments.

Finally, it should be noted that the foregoing embodiments are merely used to explain the technical solutions of the present application, but are not intended to limit the present application. Although the present application is described in detail with reference to the foregoing embodiments, the person of ordinary skill in the art should understand that they can still modify the technical solutions described in the foregoing embodiments, or make equivalent substitutions on some or all technical features therein. These modifications or substitutions do not make the essence of the corresponding technical solutions deviate from the spirit and scope of the technical solutions of the embodiments of the present application.

What is claimed is:

1. A nanoagonist, wherein a raw material of the nanoagonist comprises a transformable peptide;
the transformable peptide comprises a targeted antimicrobial peptide, a functionalized self-assembling peptide, an FcγR recognition peptide, and a lipase-responsive hydrophobic molecule that are coupled in sequence; the functionalized self-assembling peptide controls the FcγR recognition peptide to flip toward a surface of a target pathogen during secondary self-assembly, and the target pathogen is targeted and bound by the targeted antimicrobial peptide.

2. The nanoagonist according to claim 1, wherein the targeted antimicrobial peptide is used to target and bind to Gram-negative bacteria and/or Gram-positive bacteria.

3. The nanoagonist according to claim 2, wherein the targeted antimicrobial peptide is selected from the group consisting of UBI₂₉₋₄₁, a targeted antimicrobial peptide I sequence, and targeted antimicrobial peptide II sequence; and
the targeted antimicrobial peptide I sequence is shown in SEQ ID NO: 1, and the targeted antimicrobial peptide II sequence is shown in SEQ ID NO: 2.

4. The nanoagonist according to claim 3, wherein the FcγR recognition peptide comprises tuftsin.

5. The nanoagonist according to claim 4, wherein the functionalized self-assembling peptide has a sequence shown in SEQ ID NO: 3.

6. The nanoagonist according to claim 5, wherein the lipase-responsive hydrophobic molecule is one selected from the group consisting of cholesteryl hemisuccinate and monostearyl maleate.

7. The nanoagonist according to claim 6, wherein the transformable peptide has a chemical structure shown in formula [i]:

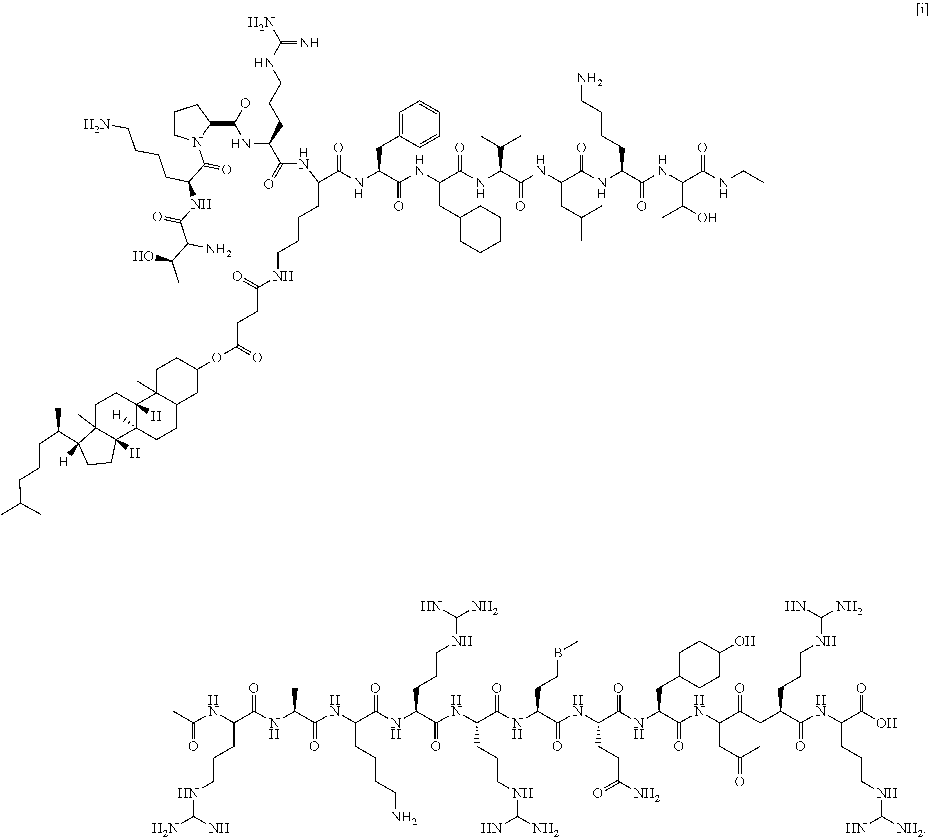

8. The nanoagonist according to claim 7, wherein the nanoagonist is a spherical nanoparticle with a particle size of 30 nm to 60 nm.

9. A preparation method of the nanoagonist according to claim 1, comprising the following steps:
synthesizing the transformable peptide;
dissolving the transformable peptide in an organic solvent to obtain a peptide stock solution; and
adding the peptide stock solution into pure water to allow self-assembly to obtain the nanoagonist.

10. A method for preparation of a drug for treating sepsis and a secondary infection thereof, comprising using the nanoagonist according to claim 1.

11. The preparation method according to claim 9, wherein the targeted antimicrobial peptide is used to target and bind to Gram-negative bacteria and/or Gram-positive bacteria.

12. The preparation method according to claim 11, wherein the targeted antimicrobial peptide is selected from the group consisting of UBI₂₉₋₄₁, a targeted antimicrobial peptide I sequence, and targeted antimicrobial peptide II sequence; and
the targeted antimicrobial peptide I sequence is shown in SEQ ID NO: 1, and the targeted antimicrobial peptide II sequence is shown in SEQ ID NO: 2.

13. The preparation method according to claim 12, wherein the FcγR recognition peptide comprises tuftsin.

14. The preparation method according to claim 13, wherein the functionalized self-assembling peptide has a sequence shown in SEQ ID NO: 3.

15. The preparation method according to claim 14, wherein the lipase-responsive hydrophobic molecule is one selected from the group consisting of cholesteryl hemisuccinate and monostearyl maleate.

16. The preparation method according to claim 15, wherein the transformable peptide has a chemical structure shown in formula [i]:

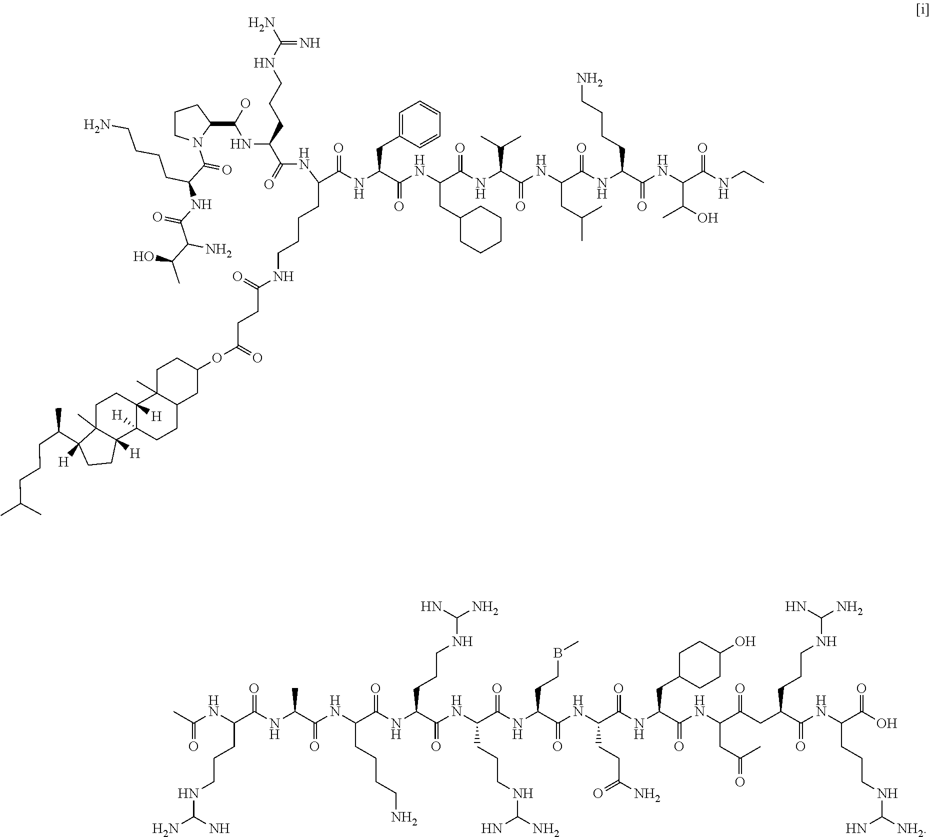

17. The preparation method according to claim 16, wherein the nanoagonist is a spherical nanoparticle with a particle size of 30 nm to 60 nm.

18. The method according to claim 10, wherein the targeted antimicrobial peptide is used to target and bind to Gram-negative bacteria and/or Gram-positive bacteria.

19. The method according to claim 18, wherein the targeted antimicrobial peptide is selected from the group consisting of UBI₂₉₋₄₁, a targeted antimicrobial peptide I sequence, and targeted antimicrobial peptide II sequence; and
the targeted antimicrobial peptide I sequence is shown in SEQ ID NO: 1, and the targeted antimicrobial peptide II sequence is shown in SEQ ID NO: 2.

20. The method according to claim 19, wherein the FcγR recognition peptide comprises tuftsin.


SEQUENCE LISTING (3 sequences)

SEQ ID NO: 1 - protein, 27 aa, synthetic construct
  source 1..27: note = Targeted antimicrobial peptide I sequence
ARGLEUTYRLEUARGILEGLYARGARG

SEQ ID NO: 2 - protein, 39 aa, synthetic construct
  source 1..39: note = Targeted antimicrobial peptide II
LYSLYSASNTYRSERSERSERILESERSERILEHISCYS

SEQ ID NO: 3 - protein, 15 aa, synthetic construct
  source 1..15: note = Functionalized self-assembling peptide
PHEPHEVALLEULYS